(19) United States
(12) Patent Application Publication
(10) Pub. No.: US 2023/0079064 A1
(43) Pub. Date: Mar. 16, 2023

(54) MOBILITY DEVICES WITH EMBEDDED SENSORS AND ASSOCIATED APPLICATION

(71) Applicant: Aligned As Designed, LLC, Los Angeles, CA (US)

(72) Inventor: Linda Smith Swerdlow, Los Angeles, CA (US)

(21) Appl. No.: 17/931,788

(22) Filed: Sep. 13, 2022

Related U.S. Application Data
(60) Provisional application No. 63/243,657, filed on Sep. 13, 2021.
(60) Provisional application No. 63/248,286, filed on Sep. 24, 2021.

(51) Int. Cl. A45B 3/08; A45B 9/04; A61H 3/02

(52) U.S. Cl. CPC A45B 3/08; A45B 9/04; A61H 3/0288; A45B 2009/007

(57) ABSTRACT

A mobility device including one or more sensors configured to transmit user mobility data to an external electronic device such as a smartphone or a computer. In embodiments, the mobility device can be a cane, crutch, walker, wheelchair, walking stick, or walking pole. A system for measuring real time mobility data of a user is also disclosed.

### RELATED APPLICATIONS

The present application claims the benefit of U.S. Provisional Application Nos. 63/243,657, filed Sep. 13, 2021, and 63/248,286, filed Sep. 24, 2021, each of which is hereby fully incorporated herein by reference.

### TECHNICAL FIELD

The present disclosure relates generally to mobility devices, including, but not limited to, crutches, canes, wheelchairs, walkers, and walking aids such as walking poles or walking sticks. More specifically, the present disclosure relates to mobility devices with embedded sensors and associated application and systems.

### BACKGROUND

Mobility devices, such as crutches, canes, wheelchairs, walkers, and walking aids, are common tools used for medical conditions that require a patient to reduce or eliminate weight on a lower extremity. Many patients require the use of mobility devices for extended periods of time. For example, many patients using crutches may have broken a bone which entails a very long healing period, and the use of little to no weight-bearing on the healing bone. Throughout the duration of using the mobility devices, many patients experience pain or discomfort from the amount of weight exerted on their hands and underarms through use of, for example, a cane or crutch.

The use of these mobility aids, whether permanent or temporary, often requires frequent trips to the doctor's office to check the injury, alignment, and use of the aids. Attempts have been made to incorporate sensors with ambulatory aids to provide patient data to medical practitioners in an effort to assist the healing process and reduce the number of trips a patient must take to the doctor's office. These past attempts have failed to sufficiently meet the needs of the patient for several reasons, including being limited in the amount and type of measurements the sensors are capable of taking, being unable to dynamically respond to changes in the patient's behavior or medical needs, and failing to address the ergonomic issues associated with traditional crutches as discussed previously.

Therefore, there remains a need for a mobility device capable of collecting and reporting real-time data to physicians and clinicians that also improves the ergonomic issues associated with traditional crutches.

SUMMARY

In embodiments, a mobility device can comprise a cane, a crutch, a walker, a wheelchair, a walking aide such as a walking stick or pole. In embodiments, the mobility device can comprise one or more devices described in one or more of U.S. Pat. Nos. 10,206,467 (Ambulatory Aid); 10,188,183 (Ambulatory Aid); 11,020,308 (Ambulatory Aid); 10,470,966 (Walker/Rollator); and U.S. Pat. App. Pub. No. 2022/0175595 (Wheelchair for Improved Muscular Skeletal System Alignment), all of which are incorporated herein by reference in their entireties. In embodiments, the mobility device includes at least one sensor positioned thereon. The at least one sensor of the mobility device can comprise any of an accelerometer, a gyroscope, a manometer, and combinations thereof In embodiments, the at least one sensor is couplable anywhere on the mobility device and/or in multiple locations. In embodiments, the sensor is repositionable, and in other embodiments, the sensor is integrated with the device such that it is not repositionable.

In some embodiments, a mobility device, such as a cane or crutch, generally comprises a shaft extending between a first end and a second end, a handle couplable to a first end of the shaft, a foot piece couplable to a second end of the shaft, and at least one sensor configured to measure mobility data from a user of the mobility device and to transmit the measured mobility data to an external electronic device, wherein the first end of the shaft is couplable to the handle such that a posterior portion of the handle extends from the shaft at a length greater than an anterior portion of the handle, and the second end of the shaft is couplable to the foot piece such that an anterior portion of the foot piece extends from the shaft at a length greater than a posterior portion of the foot portion such that the shaft extends at an angle when measured from a horizontal surface.

In embodiments, the at least one sensor of the mobility device comprises any of an accelerometer, a gyroscope, a manometer, and combinations thereof. In embodiments, the at least one sensor is couplable anywhere on the mobility device, including, but not limited to, any of one or more connectors, the handle, and the foot piece. In embodiments, the external electronic device where measured mobility data is transmitted to is a smartphone that receives the mobility data via an application. In embodiments, the mobility data is transmitted wirelessly to the external electronic device.

In embodiments, a system for measuring real time mobility data of a user generally comprises a mobility device having a shaft extending between a first end and a second end, one or more threaded connectors couplable to the shaft, a handle couplable to a first end of the shaft, a foot piece couplable to a second end of the shaft, and at least one sensor configured to measure and transmit mobility data from a user of the mobility device, and a primary external electronic device communicatively couplable to the mobility device and configured to receive mobility data from the at least one sensor, wherein the primary external electronic device uses the mobility data to create one or more dynamically-updated algorithms corresponding to a fall detection profile and a gait profile specific to the user of the mobility device.

In embodiments, the at least one sensor of the system comprises any of an accelerometer, a gyroscope, a manometer, and combinations thereof. In embodiments, mobility data taken from each of an accelerometer, a gyroscope, and a manometer are used by the primary external electronic device to create a dynamically-updated algorithm that satisfies user-specific medical parameters to create an optimal configuration for the user. In embodiments, the system for measuring real time mobility data of a user further comprises at least one secondary external electronic device communicatively couplable to the mobility device and the primary external electronic device.

The aforementioned embodiments can be used to make determinations of care for short-term injured users from one to six months and long-term disabled users. The progress or physical decline of specific groups and reduction or increase in falling and the need for medical care as related to a specific mobility device would be of value to a variety of entities including insurance companies, pharmacists, physicians, academic clinicians, hospice, and several others. The sensors will generate a stream of data every time a user engages with the disclosed devices and systems. This data will become increasingly valuable to companies active in the healthcare market and clinically relevant for patients and physicians, especially as the average age of the population increases and a larger percentage of people will require mobility devices. Early detection of gait changes will be useful to healthcare providers as part of their clinical decision making and also to advertisement sellers who market medical products.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of a mobility device according to an embodiment;

FIG. 2 is a perspective view of a foot portion of a mobility device according to an embodiment;

FIG. 3 is a perspective view of a set of crutches according to an embodiment;

FIG. 4 is a perspective view of a top half of a set of crutches according to an embodiment;

FIG. 5 is a perspective view of a closed loop cuff according to an embodiment;

FIG. 6 is a perspective view of a closed loop cuff according to an embodiment;

FIG. 7 is a perspective view of a foot portion of a set of crutches according to an embodiment.

While various embodiments are amenable to various modifications and alternative forms, specifics thereof have been shown by way of example in the drawings and will be described in detail. It should be understood, however, that the intention is not to limit the claimed disclosures to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments described herein generally pertain to mobility devices, or crutches, with embedded sensors and associated applications. Further, embodiments pertain to one or more crutches with embedded sensors that may have a closed loop cuff.

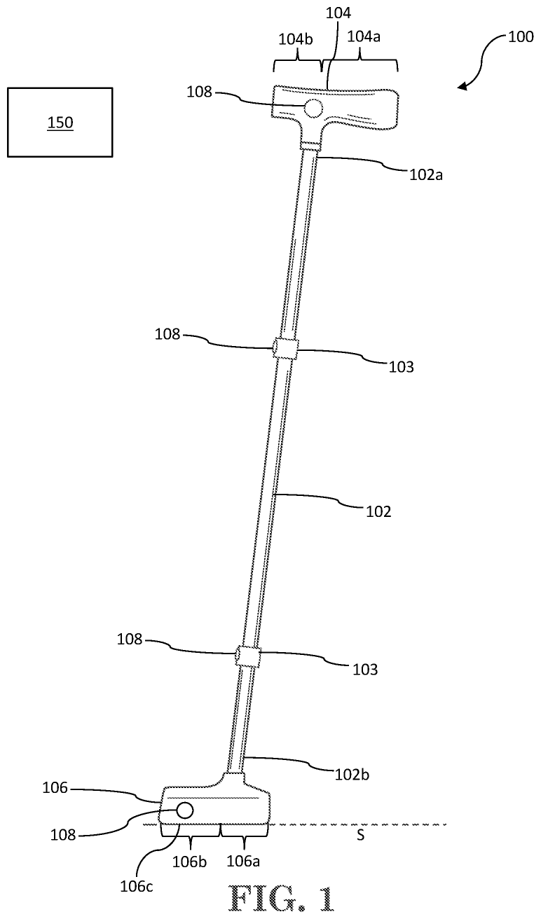

Referring to FIG. 1, according to an embodiment of the disclosure, a mobility device 100 generally comprises a shaft 102 extending between a first end 102a and a second end 102b of the shaft 102, one or more threaded connectors 103 couplable to shaft 102, a handle 104 couplable to first end 102a of shaft 102, a foot piece 106 couplable to second end 102b of shaft 102, and one or more sensors 108 couplable to various locations of mobility device 100 as desired including but not limited to threaded connector 103, handle 104, and foot piece 106.

Shaft 102 can comprise an elongate hollow, partially filled, or filled tube. Shaft 102 can comprise a cross-section that is substantially circular, oval, square, rectangular, triangular, or any of a variety of suitable shapes. Shaft 102 can be formed of lightweight aluminum, carbon fiber, plastic, or any of a variety of materials or combinations thereof, which are preferably light weight yet durable.

In embodiments, shaft 102 can be height adjustable proximate first end 102a, at a middle portion, proximate second end 102b, or any combination thereof. Shaft 102 can be height adjustable by any suitable means known to one of ordinary skill in the art including, but not limited to, a threaded connector 103 which threadably engages two portions of shaft 102 by corresponding threads formed on the connector and portions. The two portions are in telescoping arrangement such that upon loosening of the threaded connector 103, the first portion can either nest within or extend from second portion in order to shorten or lengthen, respectively, shaft 102. Upon suitable height, the threaded connector 103 is tightened by screwing. Other suitable adjustment mechanisms can include, for example, spring loaded pin(s) that are depressed, allowing the nested shaft portions to adjust relative to each other, and then released to allow the pin(s) to extend through an aperture of a series of apertures formed in shaft 102 at different heights.

In a particular embodiment, depicted in FIG. 1, shaft 102 is adjustable proximate both the first end 102a and the second end 102b of shaft 102 which allows the user to maintain postural stability and vertical alignment while adjusting the height of the mobility device 100 before sitting or standing, and before ascending or after descending a flight of stairs. In embodiments, first end 102a of shaft 102 is aligned at a more posterior angle than second end 102b of shaft 102.

In embodiments, handle 104 of mobility device 100 is elongated and extends posterior and anterior to shaft 102. A total length of handle 104 can be from about 3 inches to about 7 inches, and more particularly about 5 inches. In a particular embodiment, a posterior portion 104a of handle 104 is longer, and optionally larger in surface area, than an anterior portion 104b of handle 104 when measured from a center point of shaft 102 such that handle 104 is configured so that when the person's hand is gripping the handle, handle 104 will be offset over shaft 102. Posterior portion 104a can be from about 1.25 to about 3 times longer than anterior portion 104b. In one particular embodiment, posterior portion 104a is 1.5 times longer than anterior portion 104b, and can be, for example, about 3 inches whereas anterior portion 104b can be about 2 inches, when measured from a center point of shaft 102.

Handle 104 can be shaped similar to a handle of a traditional mobility assistance device such as a cane, including a curved top surface, or can have a more linear top surface. Handle 104 can be of any suitable material, such as an open-cell or closed-cell foam, to provide suitable support yet comfort. Handle 104 can also include an optional cover to provide additional grip, such as a silicone or rubber cover. In embodiments, a circumference or perimeter of posterior portion 104a can be equal to or greater than anterior portion 104b. In one particular embodiment, a circumference of a forward most portion of anterior portion 104b can be from about 3 inches to about 5 inches, and more particularly about 4 inches, and widens to about 3.5 inches to about 5.5 inches, and more particularly about 4.5 inches to posterior portion 104a. In embodiments, widening from anterior portion 104b to posterior portion 104a can be continuous or discrete (step-change).

Foot piece 106 of mobility device 100 is elongated and extends anterior and posterior to shaft 102. In a particular embodiment, an anterior portion 106b of foot piece 106 is longer than a posterior portion 106a. A total length of foot piece can be from about 3 inches to about 8 inches, and more particularly about 5 inches to about 6 inches, and more particularly about 5.5 inches. In a particular embodiment, anterior portion 106b of foot piece 106 is longer than posterior portion 106b of foot piece 106 when measured from a center point of shaft 102, thereby mimicking the heel and anterior portion of the foot relative to the tibia of the leg. Anterior portion 106b can be from about 1.25 to about 3.5 times longer than posterior portion 106a. In one particular embodiment, anterior portion 106b is 1.75 times longer than posterior portion 106a, and can be, for example, about 3.5 inches whereas posterior portion 106a can be about 2 inches, when measured from a center point of shaft 102.

A bottom portion 106c of foot piece 106 can be tubular or arcuate in shape. In other words, a surface S contacting bottom portion 106c of foot piece 106 is non-planar, and is curved or arcuate (circular or elliptical), allowing foot piece to roll onto and over the surface S during the gait cycle, thereby mimicking the heel to toe motion of the normal gait cycle. In embodiments, a circumference or perimeter (non-circular) varies along the length of foot piece 106, such as from about 4 inches to about 7 inches, and optionally can be wider in areas proximate shaft 102, and then tapering in both the anterior and posterior directions. In other embodiments, a circumference or perimeter of foot piece 106 is substantially constant along anterior portion 106b, posterior portion 106a, or both. A height of foot piece 106 can be larger on an end of anterior portion 106b than an end of posterior portion 106a and can range from about 1 inch to about 3 inches.

In one embodiment, foot piece 106 is formed of an interior material, such as an open-cell foam, closed-cell foam, plastic, or rubber material, and a tubular rubber or silicon exterior cover, optionally with one or more ridges formed thereon, to provide friction and additional stability.

One or more sensors 108 are couplable to various locations of mobility device 100 as desired including, but not limited to, threaded connector 103, handle 104, and foot piece 106. Sensors 108 are communicatively couplable with an external electronic device 150 configured to receive mobility data measured with sensors 108. Sensors 108 may be configured to operate with electronic hardware including memory devices, processors, power sources, transducers, transmitters, and other electronic hardware devices known to one of ordinary skill in the art. Sensors 108 are configured to collect real time mobility device 100 user data which is wirelessly transmittable to an application located on a smartphone, tablet, computer, or any other suitable external electronic device 150. User data taken from sensors 108 can then be viewed and analyzed by others such as a medical practitioner, a physical therapist, or an insurance provider. Further, user data can be uploaded to a virtual server such as a cloud server or any other similar service for subsequent storage. The user data can be assessed to provide relevant care for the user of mobility device 100 which can help decrease falling rates and improve best practices and short- and long-term outcomes for specific physical disabilities. User data may be used for a variety of practices including for fall detection and to create a gait profile of the user.

Sensors 108 may include an accelerometer, a gyroscope, and a manometer, or any combination of one or more of each type. An accelerometer can be used to measure acceleration, speed, and distance data of the mobility device 100. A gyroscope can be used to measure speed of rotation and to determine if the mobility device 100 has fallen over. A manometer can be used to measure pressure at various locations of mobility device 100 such as handle 104 which can be used to determine if the user was gripping the mobility device 100 at the time of falling over. Data from each sensor 108 can be used to create a fall detection algorithm between mobility device 100 and the external electronic device 150 application which, upon detection of an abnormal event such as the user falling over, can trigger an alert on the external electronic device 150 application. Prompt response to the alert or lack of a response from the user may trigger a separate alert to various sources such as emergency medical services to indicate that the user is in need of medical assistance. The fall detection algorithm may be dynamically updated using a continuous stream of user mobility data received from mobility device 100.

In embodiments, sensor 108 data taken from one or more of an accelerometer, a gyroscope, a manometer, or combinations thereof can be used to create a user-specific dynamically-updated gait profile. A continuous stream of sensor 108 data can assist a medical practitioner in measuring how the user's gait profile changes over time which can further be used to create predictive models using artificial intelligence that can detect early user deterioration from degenerative diseases such as Parkinson's diseases or from natural aging of the user. Further, measurement of changes in a user's gait profile over time can help medical practitioners determine if the user is experiencing ailments not directly related to mobility such as pneumonia or other flu-like illnesses which have been known to affect gait profile. In embodiments, data taken from a variety of sensors 108 including an accelerometer, a gyroscope, and a manometer can be used by the external electronic device 150 to create a dynamically-updated algorithm that satisfies user-specific medical parameters to create an optimal configuration for the user. In embodiments, multiple external electronic devices 150 may be communicatively couplable with each other and with mobility device 100. Multiple external electronic devices 150 may be divided between a primary external electronic device and one or more secondary external electronic devices.

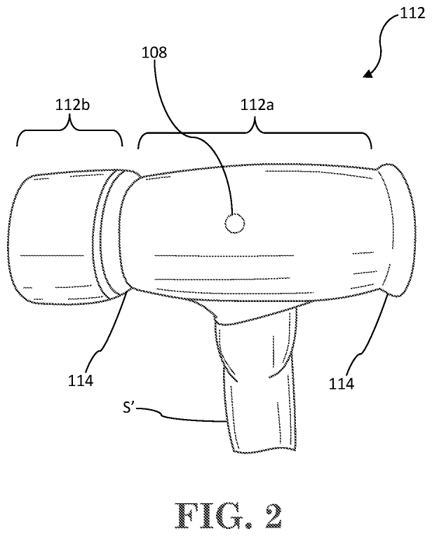

Referring now to FIG. 2, foot piece 112 according to another embodiment includes a first portion 112a having a slightly varying diameter along its length, the largest diameter occurring at a central location, and which extends both anterior to and posterior to a shaft S′ of a mobility device 100, and a second portion 112b, which has a substantially constant diameter along its length, and extends anterior to first portion 112a. A radius of curvature of second portion 112b is significantly larger than an average radius of curvature of first portion 112a, such that second portion 112b appears “flatter” than first portion 112a. A ratio of the radius of curvature of second portion 112b to first portion 112a can be in a range from about 1.25:1 to about 5:1. First portion 112a can be separated from second portion 112b by one or more ridges 114, and/or can terminate in a ridge 114. Additional ridges can be formed along first portion 112a and/or second portion 112b, either transversely and/or longitudinally as desired.

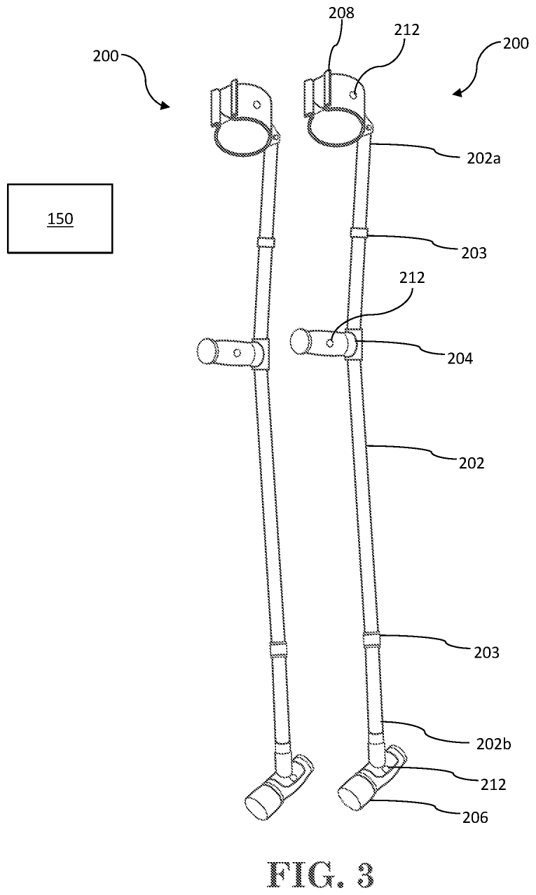

Referring now to FIG. 3, according to an embodiment of the disclosure, a crutch 200 generally comprises a shaft 202 extending between a first end 202a and a second end 202b, one or more threaded connectors 203 couplable to shaft 202, a handle 204 couplable to first end 202a of shaft 202, a foot piece 206 couplable to second end 202b of shaft 202, a closed loop cuff 208 couplable to first end 202a of shaft 202, and one or more sensors 212 couplable to various locations of crutch 200 as desired including, but not limited to, threaded connector 203, handle 204, and foot piece 206. Shaft 202 may comprise an elongate hollow, partially filled, or filled tube. Shaft 202 may be formed of lightweight aluminum, carbon fiber, plastic, or any of a variety of materials or combinations thereof, which are preferably light weight yet durable.

In embodiments, shaft 202 may be height adjustable proximate first end 202a, at a middle portion, proximate second end 202b, or any combination thereof. Shaft 202 can be height adjustable by any suitable means known to one of ordinary skill in the art. Because the height adjustment of the shaft 202 may be completed at first end 202a, middle portion, or second end 202b, the height may be manipulated to facilitate a subject sitting down, standing up, going up or down stairs, or any other movement or action that may require a height adjustment, while receiving support. Each portion of the shaft 202 may be height adjustable, via the one or more threaded connectors 203 or other suitable height adjustment mechanisms such as spring-loaded pin(s) as described previously. Height adjustment of shaft 202 may move other portions of crutch 200, such as the closed loop cuff 208, the handle 204, and the foot piece 206, relative to their original positions. Between the closed loop cuff 208 and the handle 204, the shaft 202 may extend at an angle relative to being straight. The slight angle may produce a more natural fit against an upper arm of a user of the crutch 200.

In embodiments, handle 204 extends horizontally and anterior to the shaft 202. Handle 204 mirrors the extension of foot piece 206, though foot piece 206 may be coupled to the second end 202b of shaft 202. Handle 204 may be made of any suitable material that allows for grip and comfort, including variations of plastic, rubber, or foam. Handle 204 may be different lengths or sizes based on the measurements of the hands of the user.

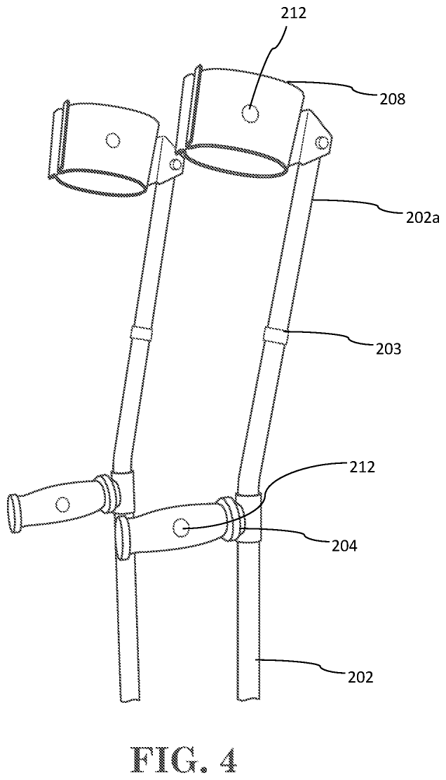

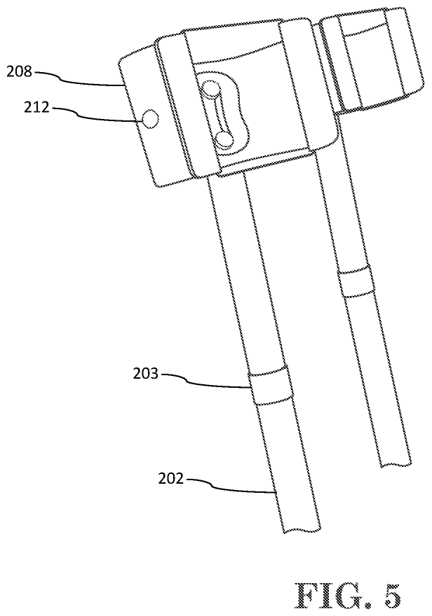

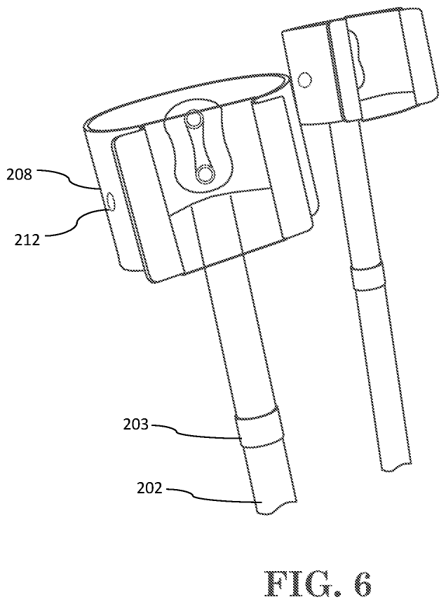

Referring now to FIG. 4, a top half of a set of crutches 200 according to an embodiment of the disclosure is depicted. Referring now to FIGS. 5 and 6, closed loop cuff 208 is preferably coupled to first end 202a of shaft 202. Closed loop cuff 208 may be removably couplable to first end 202a of shaft 202 via screws or any other suitable coupling mechanism. Closed loop cuff 208 may be fixed in size and interchangeable to fit the arm of a user of crutch 200. In another embodiment, the closed loop cuff 208 may be adjustable in size, allowing a subject to change the size to best fit the circumference of their upper arm. The posterior side of closed loop cuff 208 may have a small, straight structure to secure the closed loop cuff 208 to an arm of a user.

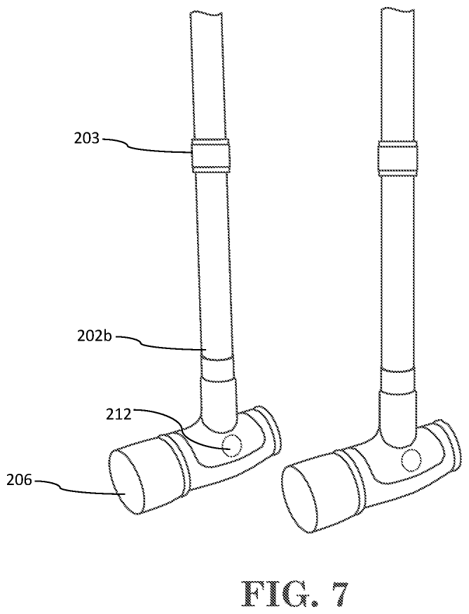

Referring now to FIG. 7, a perspective view of a foot piece 206 according to an embodiment of the disclosure is depicted. Foot piece 206 is typically circular in cross section and extends radially from second end 202b of shaft 202 about a circumference of shaft 202. In a particular embodiment, an anterior portion of foot piece 206 is longer than a posterior portion of foot piece 206 when measured from a center point of shaft 202, thereby mimicking the heel and anterior portion of the foot relative to the tibia of the leg. Foot piece 206 is generally similar to foot piece 106 in design and material.

Referring now to FIGS. 3-7, one or more sensors 212 are couplable to various locations of crutch 200 as desired including but not limited to threaded connector 203, handle 204, and foot piece 206. Sensors 212 are identical in use, design, and functionality as sensors 108 of mobility device 100. Sensors 212 may be configured to operate with electronic hardware including memory devices, processors, power sources, transducers, transmitters, and other electronic hardware devices known to one of ordinary skill in the art.

In operation, crutch 200 may have several components, including but not limited to a shaft 202, one or more threaded connectors 203, a handle 204, a foot piece 206, a closed loop cuff 208, and one or more sensors 212. Crutch 200 may come already assembled, or in separate pieces requiring assembly by a user. The closed loop cuff 208, the handle 204, and the foot piece 206 may vary in configuration, size, and material to satisfy the needs of a specific user. Sensors 212 may be removably couplable to crutch 200 so that a user can replace one or more sensors 212 with different or new sensors 212 as needed.

Various embodiments of systems, devices, and methods have been described herein. These embodiments are given only by way of example and are not intended to limit the scope of the claimed disclosures. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed disclosures.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A mobility device, and at least one sensor configured to measure mobility data from a user of the mobility device and to transmit the measured mobility data to an external electronic device.

2. The mobility device of claim 1, wherein the at least one sensor comprises any of an accelerometer, a gyroscope, and a manometer.

3. The mobility device of claim 1, wherein the mobility device comprises:
a shaft extending between a first end and a second end;
one or more threaded connectors couplable to the shaft;
a handle couplable to a first end of the shaft; and
a foot piece couplable to a second end of the shaft,
wherein at least one sensor is couplable to any of the one or more threaded connectors, the handle, and the foot piece.

4. The mobility device of claim 1, wherein the external electronic device is a smartphone configured to receive mobility data via an application.

5. The mobility device of claim 1, wherein mobility data is transmitted wirelessly to the external electronic device.

6. The mobility device of claim 1, further comprising a closed loop cuff removably couplable to the first end of the shaft.

7. The mobility device of claim 6, wherein the closed loop cuff is fixed in size.

8. The mobility device of claim 1, wherein the mobility device is selected from the group consisting of a cane, crutch, walker, wheelchair, walking stick, and walking pole.

9. The mobility device of claim 1, wherein the shaft is adjustable in at least two positions.

10. The mobility device of claim 1, wherein the shaft is adjustable in at least three positions.

11. The mobility device of claim 1, wherein the first end of the shaft is couplable to the handle such that a posterior portion of the handle extends from the shaft at a length greater than an anterior portion of the handle, and the second end of the shaft is couplable to the foot piece such that an anterior portion of the foot piece extends from the shaft at a length greater than a posterior portion of the foot portion such that the shaft extends at an angle when measured from a horizontal surface.

12. The mobility device of claim 1, wherein the foot piece has a first portion having a varying diameter along a length of the first portion which extends both anterior and posterior to the shaft.

13. The mobility device of claim 12, wherein the foot piece has a second portion which has a substantially constant diameter along a length of the second portion which extends anterior to the first portion.

14. The mobility device of claim 12, wherein the foot piece has one or more ridges that separate the first portion from the second portion.

15. A system for measuring real time mobility data of a user comprising:
a mobility device; and
a primary external electronic device communicatively couplable to the mobility device and configured to receive mobility data from the at least one sensor, wherein the primary external electronic device uses the mobility data to create one or more dynamically-updated algorithms corresponding to a fall detection profile and a gait profile specific to the user of the mobility device.

16. The system of claim 15, wherein the at least one sensor comprises any of an accelerometer, a gyroscope, and a manometer.

17. The system of claim 16, wherein mobility data taken from each of an accelerometer, a gyroscope, and a manometer are used by the primary external electronic device to create a dynamically-updated algorithm that satisfies user-specific medical parameters to create an optimal configuration for the user.

18. The system of claim 15, further comprising at least one secondary external electronic device communicatively couplable to the mobility device and the primary external electronic device.

19. The system of claim 15, wherein the mobility device comprises a shaft extending between a first end and a second end, one or more threaded connectors couplable to the shaft, a handle couplable to a first end of the shaft, a foot piece couplable to a second end of the shaft, and at least one sensor configured to measure and transmit mobility data from a user of the mobility device

20. The system of claim 15, wherein the mobility device is selected from the group consisting of a cane, crutch, walker, wheelchair, walking stick, and walking pole.
